(19) United States
(12) Patent Application Publication
(10) Pub. No.: US 2006/0282086 A1
(43) Pub. Date: Dec. 14, 2006

(54) Nasogastric tube introducer device

(76) Inventor: Mohamed Osman Abdelatti, Lancashire (GB)

Correspondence Address: MOORE & VAN ALLEN PLLC, P.O. BOX 13706, Research Triangle Park, NC, 27709, US

(21) Appl. No.: 11/067,960

(22) Filed: Feb. 28, 2005

(51) Int. Cl. A61F 11/00

(52) U.S. Cl. 606/108

(57) ABSTRACT

A device for introducing a nasogastric tube in the stomach of anaesthetised or comatose patients by medical personnel is described. It consists of a curve-shaped plastic or rubber tube. It has two extensions 2, 3 at its proximal end 4 for holding it and to prevent it from being dislodged into the oesophagus during insertion of the NGT and a hair-line slit 5 along its side extending from its proximal end 4 to the distal end 6 for separating it from the nasogastric tube. The depth of the slit in the wall of the introducer is partially glued to prevent the tip of the nasogastric from leaving the introducer via the slit during insertion while allowing the nasogastric tube to be easily peeled off the introducer.

### FIELD OF THE INVENTION

This invention relates to a device to assist anaesthetists with insertion of a nasogastric tube (NGT) in the stomach of an anaesthetised or comatose patient.

### BACKGROUND TO THE INVENTION

The insertion of a nasogastric (stomach) tube (NGT) is an important procedure that is routinely performed in the daily practice of anaesthesia for emergency and elective abdominal surgical operations. All ventilated patients in the intensive care unit have a nasogastric tube placed for therapeutic, diagnostic and or feeding. The NGT is a narrow—bore soft plastic/rubber tube. It is usually inserted via the nose into the mouth and then it is advanced, blindly, so that its tip lies in the stomach. One aim of the NGT is to empty the stomach of secretion and prevent abdominal distension in intestinal obstruction. Another aim of the NGT is to instil drugs or feeding material into the stomach. A further aim of the NGT is to empty the stomach of air/anaesthetic gases because a distended stomach may be damaged during blind insertion of equipments essential for laparoscopic (keyhole) surgery e.g. removal of the gall bladder.

However, in practice, the presence of a tracheal tube in the oral cavity makes the insertion of the pliable NGT (stomach tube) difficult because the latter tends to curl in the mouth instead of passing into the stomach. To overcome this problem, the NGT can be cooled before use to stiffen it but the tube tends to warm up quickly and become soft again once it is introduced in the mouth. Another method of inserting a NGT is to use a laryngoscope to visualize the tip of the NGT once it enters the mouth from the nose and to employ a forceps to grasp its tip and advance it into the stomach. This repeated grasping of the tube to advance it in the stomach leads to injury of the oral mucosa (lining) and severe sore throat after the operation.

The usual practice, if the above techniques fail, is to grasp the tip of the NGT once it appears in the mouth cavity with a forceps and draw it out of the mouth. Then select a tracheal tube with a diameter larger than the NGT and split it along its side. This modified tracheal tube is then passed through the mouth into the oesophagus. The tip of NGT is threaded into this tube and advanced into the stomach. The NGT is then separated from the tracheal tube along the slit and the tracheal tube is removed from the oesophagus.

Although this technique is very successful, the use of a modified tracheal tube for this purpose has several disadvantages. One disadvantage is the fact that time is wasted in preparing and cutting the tracheal tube to adjust its length and to create a slit along its side. Another disadvantage is the fact that the modified tracheal tube also has no means for holding it outside the mouth and can easily be lost into the oesophagus. A further disadvantage is the fact that the manufactures of tracheal tubes may deny responsibility for any damage/complications caused by modification of their tracheal tubes and their use for a different purpose than what they are intended for i.e. for ventilation and not as guide to the NGT.

This indicates that there is a need for a specifically designed, simple, safe to use and readily available NGT-I because currently, there is no commercially available one.

It is an aim of the present invention to overcome at least one problem associated with the prior art whether referred to herein or otherwise.

### SUMMARY OF THE INVENTION

According to a first aspect of the present invention there is provided a nasogastric tube introducer device comprising guide means in order to guide the nasogastric tube, the guide means comprising a longitudinal conduit having a peripheral wall defining a passageway therethrough extending from a first end to a second end, the conduit comprising a longitudinal releasing region which enables a slit to be defined in the peripheral wall of the conduit in order to enable the nasogastric tube to be moved radially relative to the conduit in order to be moved out of the passageway defined by the conduit.

Preferably the longitudinal releasing region comprises a slit which is secured together along at least a part of the longitudinal length thereof and preferably along the full longitudinal length thereof.

The longitudinal releasing region may comprise a slit which is adhered together along at least a part of the longitudinal length thereof and preferably along the full longitudinal length thereof.

The longitudinal releasing region may comprise a slit which is sealed, bonded or otherwise secured along at least a part of the longitudinal length thereof and preferably along the full longitudinal length thereof.

The longitudinal releasing region may comprise a length or strip of securement means. The securement means may comprise an adhesive and preferably comprises glue.

Preferably the peripheral wall of the conduit is secured together along at least a part of the longitudinal releasing region and may form a substantially annular cross-section. More preferably the peripheral wall of the conduit is secured together around the periphery along the full longitudinal releasing region.

Preferably, prior to use, a slit is not defined through the peripheral wall of the conduit along a part or the full longitudinal length of the conduit.

The peripheral wall may be adhered along the longitudinal releasing region.

The longitudinal releasing region may comprise a relatively weakened longitudinal region defined along the peripheral wall of the conduit.

The longitudinal releasing region may comprise an adhesive and preferably comprises a strip of an adhesive.

Preferably the thickness of the longitudinal releasing region is less than the thickness of the wall of the conduit. The thickness of the longitudinal releasing region may be approximately 50% of the thickness of the wall of the conduit.

Preferably the conduit may be split along the longitudinal releasing region and preferably is split to enable a nasogastric tube to be radially moved from within a passageway of the conduit in order for the conduit to be removed from around the nasogastric tube.

Preferably, the longitudinal releasing region extends for substantially the full longitudinal length of the conduit.

Preferably the longitudinal releasing region extends for the complete length of the conduit.

Preferably the longitudinal releasing region comprises a continuous securing region along the length of the conduit.

Preferably the longitudinal releasing region extends from the first longitudinal end to the second longitudinal end of the conduit.

The longitudinal releasing region may be separable from the conduit. The longitudinal releasing region may be peelable or otherwise removable from the conduit and this may cause a slit to be exposed or defined in the conduit.

The longitudinal length of the conduit may be greater than 10 cm. The longitudinal length of the conduit may be substantially 15 cm. The longitudinal length of the conduit may be substantially 25 cm. The longitudinal length of the conduit may be less than 30 cm.

The internal diameter of the conduit may be greater than 2 mm. The internal diameter of the conduit may be substantially 4 mm. The internal diameter of the conduit may be substantially 8 mm. the internal diameter of the conduit may be less than 10 mm.

Preferably the conduit is arcuate or curved.

Preferably the conduit comprises a plastics.

Preferably the longitudinal releasing region comprises a slit in the peripheral wall of the conduit which has been secured or adhered together. Preferably said slit is adhered by an adhesive wherein the thickness of the adhesive is less than the thickness of the peripheral wall of the conduit. The strength of the means for sealing the slit is preferably less than the strength of the material of the peripheral wall of the conduit.

Preferably the lateral cross-section of the conduit and the longitudinal releasing region defines a continuous or contiguous passageway wall and preferably an inner passageway wall.

Preferably the longitudinal releasing region comprises a hair line slit which is temporarily secured together or sealed.

The device may comprise indicator means to indicate the location of the longitudinal releasing region. The indicator means may comprise a channel portion defined along the peripheral wall of the conduit and preferably said channel overlies the longitudinal releasing region.

Preferably, in use, the first end locates outside the mouth cavity of the patient.

Preferably, in use, the second end locates within the mouth cavity of the patient. Preferably, in use, the second end locates in or towards the oesophagus of the patient.

Preferably the device comprises retaining means to retain at least a part of the device outside the mouth cavity of the patient.

The retaining means may be located at or towards the first longitudinal end of the conduit.

The retaining means may comprise a first retaining member which is arranged, in use, to contact or abut a part of the patient around the mouth area to prevent the device (and specifically the first end of the device) from entering or fully entering the mouth cavity of the patient.

The retaining means may comprise a first retaining member and a second retaining member which are arranged, in use, to contact or abut a part of the patient around the mouth area to prevent the device (and specifically the first end of the device) from entering or fully entering the mouth cavity of the patient.

The or each retaining member may comprise a resilient material.

The device may comprise grip means to enable an operative to grip the device and preferably enables an operative to grip the device during insertion of the nasogastric tube through the device and preferably into the stomach of the patient.

The grip portion may comprise a handle and preferably comprises a spoon shaped handle.

Preferably the nasogastric tube introducer device is arranged to be peeled off the nasgastric tube.

According to a second aspect there is provided an assembly comprising a nasogastric tube and a nasogastric tube introducer device wherein the nasogastric tube introducer device is in accordance with the first aspect of the present invention.

According to a third aspect of the present invention there is provided a method of introducing a nasogastric tube into the stomach of a patient comprising inserting a nasogastric tube through a conduit of a nasogastric tube introducer device and subsequently opening a longitudinal releasing region in order to define a slit in a peripheral wall of the conduit to radially move the nasogastric tube relative to the conduit in order to remove the nasogastric tube from a passageway defined in the conduit.

The method may comprise separating the peripheral wall of the conduit to define a slit.

According to another aspect of the present invention there is provided a nasogastric tube introducer device for assisting the insertion of a nasogastric tube into the stomach of an anaesthetised patient is comprised of a curved hollow tube with two extensions at its proximal end and a hair-line slit extending from its proximal end to its distal end such that the slit in the wall of the tube is partially closed by a film of glue along half of the inner depth of the slit and the remaining outer half of the slit is left open to help mark the position of the slit.

The curved tube may have two extensions at its proximal end, the first extension may be spoon shaped to facilitate holding the tube and the second extension may be shorter than the first extension.

### BRIEF DESCRIPTION OF THE DRAWINGS

The present invention and a specific embodiment of the device will now be described by way of example only, with reference to the accompanying drawings that follow, in which:

FIG. 1 is a schematic diagram of an embodiment of an NGT introducer device.

FIG. 2 is a cross section of an embodiment of an NGT introducer device showing that half of the depth of the slit is glued.

FIG. 3 is a schematic diagram to illustrate the technique of insertion of an embodiment of an NGT using an embodiment of a NGT introducer device.

### DESCRIPTION OF THE PREFERRED EMBODIMENTS

The nasogastric tube (NGT) introducer device 10 comprises a conduit 12 providing a passageway 16 therethrough. The conduit comprises an elongate tube which extends longitudinally from a first longitudinal end 20 (the proximal end) to a second longitudinal end 18 (the distal end) or tip. In use, the conduit 12 is inserted into the mouth of a patient in order for the proximal end 20 to locate outside the mouth of the patient and for the distal end 18 to locate in or towards the oesophagus of the patient. The NGT 11 can then be easily inserted into the passageway 16 of the proximal end 20 of the conduit 12 and the NGT is easily guided down into the oesophagus of the patient.

The conduit 12 comprises a longitudinal releasing portion in the form of a separable portion 14 extending longitudinally along the conduit 12. The separable portion 14 enables the periphery of the conduit to be separated in order to define a slit along the longitudinal length of the peripheral wall of the conduit 12 in order for the NGT 11 to be easily removed from within the passageway 16 of the conduit 12. In the preferred embodiment, the separable or releasing portion 14 extends longitudinally along the full longitudinal length of the conduit 12 from the proximal end 20 to the distal end 18. However, since the peripheral walls of the conduit 12 defining the passageway 16 are initially secured this prevents and inhibits the NGT 11 from emerging through the separable portion 14 whilst the NGT 11 is being pushed through the conduit 12.

The peripheral wall of the conduit, prior to use, does not have a slit defined along the length thereof. The peripheral wall of the conduit is continuous or contiguous around the circumference thereof. The longitudinal releasing means comprises a slit which has been secured together to prevent inadvertently opening of the slit. In particular, the slit is adhered together with a glue along the length thereof.

The conduit 12 comprises a curved hollow tube which is shaped in order to enable the NGT 11 to be easily inserted therethrough and also is shaped to accurately locate the distal end 18 in or towards the oesophagus of the patient.

The conduit 12 comprises a tube which has been slit along the length thereof to define a longitudinal slit. In one embodiment, the walls of the conduit 12 forming the slit are secured to each other by adhesive means and preferably with a glue 22. The depth of the glue 22 may be half the thickness of the wall 24 of the conduit 12 and this enables the conduit 12 to be easily split when desired but prevents or inhibits inadvertent splitting of the conduit 12.

In use, the end of the NGT 11 is inserted through the nose of the patient and enters the mouth cavity. The operator or user can then remove the end of the NGT 11 from the mouth cavity. The NGT introducer device 10 is inserted and correctly placed in the mouth cavity of the patient. The end of the NGT can then be inserted through the conduit 12 and down into the stomach of the patient. Once correctly positioned, the conduit 12 can be opened along the slit 14 in order to release the section of the NGT 11 from the conduit 12 and the NGT introducer device can be removed from the mouth cavity and is no longer required.

The device 10 comprises location identifying means to identify or mark the location of the slit or separable portion 14. The location may be identified by the presence of a channel in the wall 24 of the conduit 12. Such a channel may be formed by the fact that the thickness of the slit 14 is less than the thickness of the wall 24 of the conduit 12. Specifically, the internal passageway surface of the conduit 12 is smooth and thereby prevents the tip of the NGT from snagging any irregularities in the surface and, since the slit has a lower thickness this difference in thickness causes a channel to be formed longitudinally along the outer surface of the wall 24 of the conduit 12.

The device 10 comprises grip means to enable the device to be manually gripped by an operative. The grip means may comprise a handle in the form of an extension 26 located at or towards the proximal end 20. The grip means may be gripped by a single hand of the user, for example, between a thumb and finger of the user. This enables the user to control the piston of the device 10 and helps to aid the passage of the NGT through the passageway 16. The grip means is spoon shaped.

The device 10 also includes retaining means to retain at least the proximal end 20 outside the mouth cavity of the patient. This thereby prevents or inhibits the device 10 from entering the mouth cavity and moving down into the oesophagus. The retaining means comprises an abutment member 28 which is arranged to contact or abut a part of the patient surrounding the mouth opening. The abutment member comprises an extension 28 or extending portion.

The extension is slightly shorter than the spoon shaped grip means 26.

The grip means 26 may also help in retaining the proximal end 20 of the device 10 outside of the mouth cavity. The extension 28 extends outwardly from the proximal end 20 at an angle about the longitudinal axis of the conduit 12 relative to the grip means 26. In particular, the extension 26 may be at 180° about the longitudinal axis relative to the grip means 28.

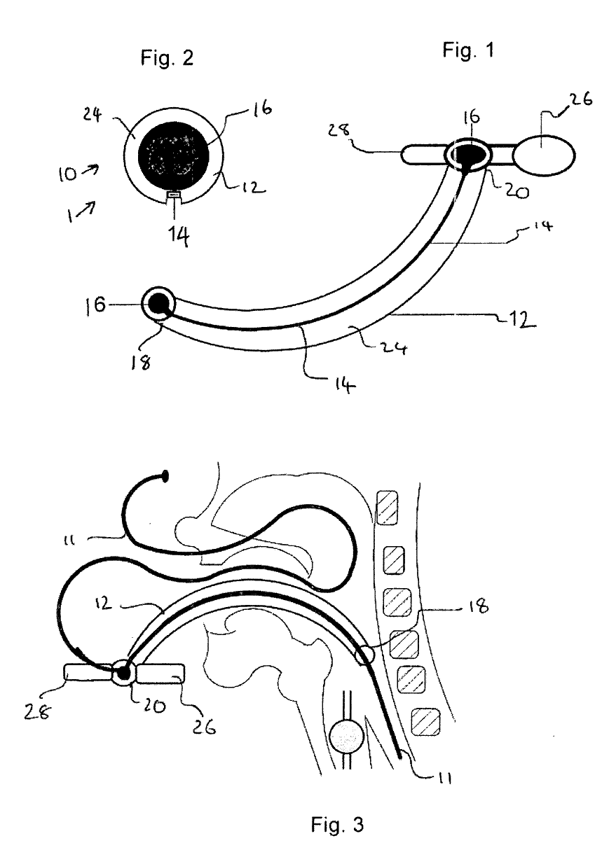

As shown in FIGS. 1 to 3, there is provided an NGT introducer 11 comprising a curve-shaped, 25 cm long, 8 mm internal diameter plastic or rubber tube 12 for adults (15 cm long, 4 mm internal diameter for children). The tube 12 has a hair-line side slit 14 5 that extends from its tip 20 6 to the proximal end 184. The slit 14 in the wall 24 of the tube 12 is incomplete (as shown in FIG. 2). Half of the depth of the slit 14 in the wall of the introducer is glued and the remaining half is left open as a partial slit 14. This prevents the nasogastric tube 11 from leaving the introducer 10 while allowing separation of the nasogastric tube 11 off the introducer 10 by peeling open the glued 14 part of the introducer 11. The NGT introducer 11 also has two extensions 26,282, 3 at the proximal end 20. One extension 26 of the tube is longer than the other extension 25 and has a spoon-shaped handle to assist holding the introducer 10 during insertion of the nasogastric tube 11 into the stomach. The other extension 28 is shorter and its purpose is to prevent the NGT introducer 10 from being lost into the oesophagus during insertion of the nasogastric tube 10.

The introducer tube 12 can be made of any reasonable material e.g. soft plastic, polyvinyl chloride or even rubber. Various sizes of tubes 12 can also be designed for use in children e.g. (15 cm long, 4 mm internal diameter) and (25 cm long, 8 mm internal diameter) for adults.

The nasogastric tube introducer device 10 assists medical personnel in the safe and easy insertion of a nasogastric tube 11 in the stomach of an anaesthetised patient. The device 10 comprises a curved hollow tube 12 made of any appropriate material e.g. rubber, polyvinyl chloride or plastic. The device 10 has two extensions 26, 28 at its proximal end 20 and a hair-line slit 14 in its wall 24 extending from its proximal end to the distal end 18. The slit 14 is partially closed by application of a film of glue 22 along half of the depth of the slit 14 in the wall 24 of the introducer 10 to prevent or inhibit the nasogastric tube tip from leaving the introducer tube 12 during insertion. The remaining outer half of the slit 14 is left open to help mark the position of the slit 14 and allow the introducer tube 12 to be easily separated from the nasogastric tube 11.

One of the extensions 26 at the proximal end 20 of the introducer is spoon-shaped to facilitate holding the introducer tube 12 during insertion of the nasogastric tube 11 and the second extension 28 is shorter but protrudes beyond the mouth opening to prevent the introducer tube 12 from it dislodgement into the mouth cavity and/or its disappearance into the oesophagus during insertion.

The device 10 is constructed from any material compatible with its use in clinical practice e.g. rubber, polyvinyl chloride or plastic wherein the size of the introducer tube 12 ranges between 15 cm long, 4 mm internal diameter for children and 25 cm long, 8 mm internal diameter for adults.

The present invention provides a device 10 for the insertion of a nasogastric tube 11 in to the stomach of an anaesthetised or comatose patient directly via the nose and or via the mouth by medical personnel

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A nasogastric tube introducer device comprising guide means in order to guide the nasogastric tube, the guide means comprising a longitudinal conduit having a peripheral wall defining a passageway therethrough extending from a first end to a second end, the conduit comprising a longitudinal releasing region which enables a slit to be defined in the peripheral wall of the conduit in order to enable the nasogastric tube to be moved radially relative to the conduit in order to be moved out of the passageway defined by the conduit.

2. A nasogastric tube introducer device according to claim 1 in which the longitudinal releasing region comprises a slit which is secured together along at least a part of the longitudinal length thereof.

3. A nasogastric tube introducer device according to claim 1 in which the longitudinal releasing region comprises a slit which is secured together along the full longitudinal length thereof.

4. A nasogastric tube introducer device according to claim 1 in which the longitudinal releasing region comprises a slit which is adhered together along at least a part of the longitudinal length thereof.

5. A nasogastric tube introducer device according to claim 1 in which the longitudinal releasing region comprises a slit which is sealed, bonded or otherwise secured along at least a part of the longitudinal length thereof.

6. A nasogastric tube introducer device according to claim 1 in which the longitudinal releasing region comprises a length or strip of securement means.

7. A nasogastric tube introducer device according to claim 6 in which the securement means comprises an adhesive.

8. A nasogastric tube introducer device according to claim 1 in which the longitudinal releasing region comprises a relatively weakened longitudinal region defined along the peripheral wall of the conduit.

9. A nasogastric tube introducer device according to claim 1 in which the thickness of the longitudinal releasing region is less than the thickness of the wall of the conduit.

10. A nasogastric tube introducer device according to claim 9 in which the thickness of the longitudinal releasing region is approximately 50% of the thickness of the wall of the conduit.

11. A nasogastric tube introducer device according to claim 1 in which the longitudinal releasing region extends from the first longitudinal end to the second longitudinal end of the conduit.

12. A nasogastric tube introducer device according to claim 1 in which the longitudinal releasing region is separable from the conduit.

13. A nasogastric tube introducer device according to claim 1 in which the conduit is arcuate or curved.

14. A nasogastric tube introducer device according to claim 1 in which the conduit comprises a plastics.

15. A nasogastric tube introducer device according to claim 1 in which the longitudinal releasing region comprises a hair line slit which is temporarily secured together or sealed.

16. A nasogastric tube introducer device according to claim 1 in which the device comprises indicator means to indicate the location of the longitudinal releasing region.

17. A nasogastric tube introducer device according to claim 16 in which the indicator means comprises a channel portion defined along the peripheral wall of the conduit.

18. A nasogastric tube introducer device according to claim 1 in which, in use, the first end locates outside the mouth cavity of the patient.

19. A nasogastric tube introducer device according to claim 1 in which, in use, the second end locates within the mouth cavity of the patient.

20. A nasogastric tube introducer device according to claim 19 in which, in use, the second end locates in or towards the oesophagus of the patient.

21. A nasogastric tube introducer device according to claim 1 in which the device comprises retaining means to retain at least a part of the device outside the mouth cavity of the patient.

22. A nasogastric tube introducer device according to claim 21 in which the retaining means is located at or towards the first longitudinal end of the conduit.

23. A nasogastric tube introducer device according to claim 21 in which the retaining means comprises a first retaining member which is arranged, in use, to contact or abut a part of the patient around the mouth area to prevent the device from fully entering the mouth cavity of the patient.

24. A nasogastric tube introducer device according to claim 1 in which the device comprises grip means to enable an operative to grip the device.

25. A nasogastric tube introducer device according to claim 24 in which the grip portion comprises a handle.

26. (canceled)

27. A method of introducing a nasogastric tube into the stomach of a patient comprising inserting a nasogastric tube through a conduit of a nasogastric tube introducer device and subsequently opening a longitudinal releasing region in order to define a slit in a peripheral wall of the conduit to radially move the nasogastric tube relative to the conduit in order to remove the nasogastric tube from a passageway defined in the conduit.

28-30. (canceled)
